(19) United States
(12) Patent Application Publication
(10) Pub. No.: US 2009/0018417 A1
(43) Pub. Date: Jan. 15, 2009

(54) APPARATUS MONITORING SIGNAL IN SITU

(76) Inventor: Wei-Kung WANG, Taipei (TW)

Correspondence Address: LADAS & PARRY LLP, 26 WEST 61ST STREET, NEW YORK, NY, 10023, US

(21) Appl. No.: 12/173,275

(22) Filed: Jul. 15, 2008

Related U.S. Application Data
(63) Continuation-in-part of application No. 10/924,021, filed on Aug. 23, 2004, now abandoned.
Application No. 10/924,021 is a continuation-in-part of application No. 10/123,124, filed on Apr. 16, 2002.
Application No. 10/123,124 is a continuation-in-part of application No. 09/766,237, filed on Jan. 19, 2001, now abandoned.

(30) Foreign Application Priority Data
Aug. 27, 2003 (TW) 092123724

(51) Int. Cl. A61B 5/1455; A61B 5/145

(52) U.S. Cl. 600/316; 600/309; 600/322; 600/344

(57) ABSTRACT

For repeatedly measuring signals from a fixed position of a tissue to monitor the blood composition, we use one or more elastic membranes at upper and lower parts of the extruded tissue together with a cone-shaped guide. This will constrain the tissue in the fixed position when a signal guide is used for measuring signals from the fixed position of the tissue repeatedly. The signals can be from an aggregate of the designated composition with the other ingredients of the blood.

### CROSS-REFERENCE TO RELATED APPLICATIONS

This application is a continuation-in-part of copending application Ser. No. 10/924,021 filed on Aug. 23, 2004, entitled “AN APPARATUS MONITORING SIGNAL IN SITU”, which is Continuation-In-Part of application Ser. No. 10/123,124 filed on Apr. 16, 2002 which is Continuation-In-Part of application Ser. No. 09/766,237 filed on Jan. 19, 2001 (now abandoned) claims the benefit thereof and incorporates the same by reference.

### BACKGROUND OF THE INVENTION

(A) Field of the Invention

This invention relates to medical devices and blood composition sampling and bio-secure systems.

(B) Description of Related Art

U.S. application Ser. Nos. 10/123,124 and 10/207,610

### SUMMARY OF THE INVENTION

There is a need to repeatedly measure the most important physiological parameters, such as blood sugar, blood oxygen and cholesterol, in order to monitor the variations thereof. For such a purpose, it is provided with a signal generator in the tissue, for example, a radio-isotope in the tissue emits a signal such as α, β, γ particles. Also, the signal can be an electromagnetic wave (visible light, UV, IR, X-ray, microwave) from outside the tissue. After the tissue is irradiated, absorption, scattering, fluorescence, etc., are induced in the tissue. A signal analyzer or a spectrum analyzer may be used to monitor the concentration of ingredients in the tissue through monitoring an induced signal from the tissue. The induced signal may not be from the ingredient itself. The induced signal may also be from an aggregate of the ingredient with some other specific component, such as

Aggregate←→ingredient (to be measured)+specific component.

In the invention, glucose and hemoglobin are used as an example.

Hemoglobin+glucose←→HbAlc (precursor)←→HbAlc

Before hemoglobin and glucose become a compound HbAlc, there is an intermediate stage of aggregate HbAlc (precursor). As the concentration of Hb is somewhat stable, we may figure out the concentration of glucose in the blood from the signal of HbAlc (precursor).

To fix the tissue at the same position for sequential measurements, a tissue adaptor is invented. The tissue adaptor works better with an extruded tissue, such as a finger or toe. If there is a cone-shaped guide inside the tissue adaptor, it guides the finger to anchor at the top of the cone-shaped guide in operation. To secure the finger in a stretched position and at the right angle, soft pads both above and below the finger can be used. These soft pads could be replaced with one or more elastic membranes which are stretched to wrap around the finger and hold the finger smoothly and softly so that no blood circulation is interfered. These pads or membranes can be in a concaved slot. The above entire structure is called the tissue adaptor. One or more springs can be used to hold the tissue adaptor to improve its adaptability.

To improve the precision of positioning the finger, a patient's palm may be placed on a flat pad, so that the finger does not rotate due to incorrect posture. For security purposes, in U.S. patent application Ser. No. 10/207,610, entitled “BIOSECURE METHOD AND DEVICE,” the cone-shaped guide of the tissue adaptor extends to cover most parts of a finger and to fit tightly with the finger, so that fingers with different shapes cannot fit into the shape. However, if the finger is too large, it cannot enter the designated slot, and if too small, it cannot be stable.

### BRIEF DESCRIPTION OF THE DRAWINGS

FIG. 1 is a cross-sectional view of an embodiment of the apparatus for monitoring a signal in situ of the present invention.

FIG. 2 is a top view of the embodiment of the apparatus for monitoring a signal in situ of the present invention as shown in FIG. 1.

FIG. 3 is a partially enlarged view of FIG. 1.

### DETAILED DESCRIPTION OF THE INVENTION

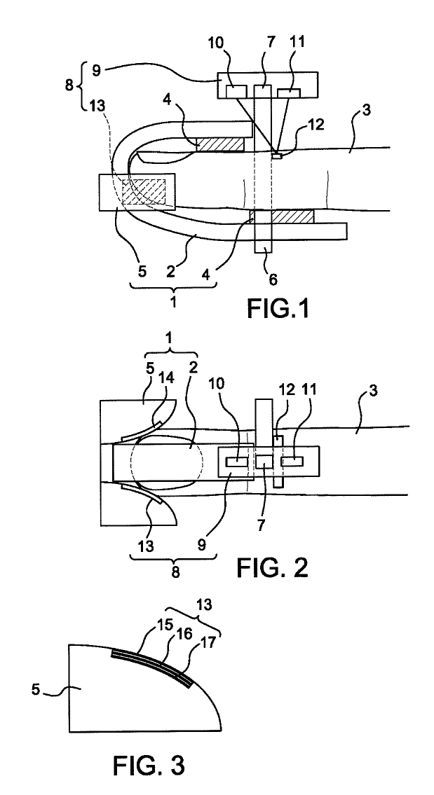

FIGS. 1 and 2 respectively are a cross-sectional view and a top view of an embodiment of the apparatus for monitoring a signal in situ of the present invention. The apparatus comprises a tissue adaptor 1 for fixing tissue for repeated measurements during repeated insertions. A concaved slot 2 is made with a mold in the shape of an extruded tissue, here a finger 3. The concaved slot 2 and the finger 3 are complementary in shape. Soft pads 4 are located inside the slot and both above and below the finger 3 so as not to interfere signals. A cone-shaped guide 5 is located at the distal end inside the concaved slot 2 to insure the correct position of the finger 3. The apparatus has a signal generator 6, positioned under the finger 3, for generating a signal to be transmitted to a fixed position of the finger 3 via a hole through the concaved slot 2 and one of the soft pads 4, and a signal analyzer 7, positioned opposite to the signal generator 6 with respect to the finger 3, for receiving and analyzing an induced signal from the fixed position of the finger 3 (i.e. for receiving and analyzing an induced signal from the finger 3 in situ). The signal generator 6 and the signal analyzer 7 are connected together in order to fix their relative position. With the help of the cone-shaped guide 5, the finger 3 can be fixed in order make a new measurement on the fixed position of the finger 3 where previous measurements were made. Thereby, repeated measurements, i.e. the so-called “monitoring,” could be made on the fixed position of the finger 3 during repeated insertions of the finger 3. It is pointed out in the invention that the induced signal used for composition analysis of a special ingredient is generated by the ingredient-making chemical actions with other ingredients in blood.

To improve the precision of positioning the finger 3, a patient's palm may be placed on a flat pad, so that the finger 3 does not rotate due to incorrect posture. The cone-shaped guide 5 extends to cover some part of the finger 3 and fit with the finger 3, so that fingers with different shapes stop at a specific position. If the finger 3 is large, it enters less deep in the concaved slot 2, and if the finger 3 is small, it enters deeper in the concaved slot 2.

The apparatus may comprise a position-fixing device 8 for fixing the finger 3 more precisely. The position-fixing device 8 may comprise a moving component 9 to refine the position of the finger 3 precisely. The moving component 9, attached with a second signal generator 10, a detector 11 and the signal analyzer 7 as described above, is positioned above the finger 3 and moves relatively to the finger 3. The second signal generator 10 generates a second signal, such as light, etc., to be transmitted to a marker 12. The marker 12 can be a natural one such as an edge, the nail, and a wrinkle of the finger 3, etc., or an artificial one painted or pasted on the finger 3. When reflected signal of the marker 12 is detected by the detector 11, the reflected signal informs the apparatus the position of the marker 12. The apparatus then knows the precise position of the finger 3 and thereby moves the detector 11, attached to the moving component 9, to the position where the reflected signal of the marker 12 can be detected by the detector 11. In this way, the position of the finger 3 may be positioned more precisely. The position-fixing device 8 may further comprise a three-element switch 13 (see FIG. 3 for the enlarged view of the three-element switch 13) coupled with the cone-shaped guide 5 to help fixing the position of the finger 3 even more precisely. The three-element switch 13 is off when all three elements 15, 16, 17 contained therein are isolated. When the element 15 touches the element 16, the three-element switch 13 is turned “ON.” When all the three elements 15, 16, 17 touch with each other, the three-element switch 13 is turned “OFF.” The finger 3 is thereby confined in between the elements 15 and 16 to ensure that the three-element switch 13 remains “ON” during repeated measurements. The position-fixing device 8 may further comprise an additional three-element switch 14 coupled with the cone-shaped guide 5 to help fixing the position of the finger 3. When the finger 3 is moved away either way from the fixed position, the two three-element switches 13 and 14 are turned off.

What is claimed is:

1. An apparatus for monitoring an induced signal in situ comprising:
a signal generator;
a signal analyzer; and
a tissue adaptor to fix the tissue in situ during repeated measurements,
wherein a signal generated by the signal generator is transmitted to the tissue, and the induced signal from the tissue in situ is received and analyzed by the signal analyzer.

2. An apparatus as claimed in claim 1, wherein the induced signal from the tissue is from an ingredient in the tissue.

3. An apparatus as claimed in claim 2, wherein the ingredient comprises aggregate.

4. An apparatus as claimed in claim 3, wherein the aggregate comprises glucose.

5. An apparatus as claimed in claim 3, wherein the aggregate comprises hemoglobin.

6. An apparatus as claimed in claim 3, wherein the aggregate comprises both glucose and hemoglobin.

7. The apparatus as claimed in claim 1, wherein the signal comprises an electromagnetic wave.

8. The apparatus as claimed in claim 1, wherein the tissue comprises an extruded shape.

9. The apparatus as claimed in claim 1, wherein the tissue adaptor comprises soft pads.

10. The apparatus as claimed in claim 1, wherein the tissue adaptor comprises a cone-shaped guide to confine the tissue.

11. The apparatus as claimed in claim 1, wherein the tissue adaptor comprises a concaved slot fit closely to the tissue.

12. The apparatus as claimed in claim 1, wherein the tissue adaptor comprises a spring.

13. The apparatus as claimed in claim 1, wherein the tissue adaptor comprises an elastic membrane.

14. The apparatus as claimed in claim 1, further comprising a position-fixing device.

15. The apparatus as claimed in claim 14, wherein the position-fixing device comprises a moving component, the moving component, attached with a second signal generator and a detector, moves relatively to tissue to a fixed position during repeated measurements,

16. An apparatus as claimed in claim 15, wherein the second signal generator generates a second signal to be transmitted to a marker on the tissue and a signal detector for detecting reflected signal of the marker on the tissue.

17. An apparatus as claimed in claim 16, wherein the marker is a natural one or an artificial one.

18. An apparatus as claimed in claim 14, wherein the position-fixing device comprises a three-element switch coupled with the cone-shaped guide to further define the position of the tissue.

19. An apparatus as claimed in claim 18, wherein the three-element switch is turned on when the tissue touches it at a second fixed position and is turned off when the tissue touches it in a position other than the second fixed position.

20. An apparatus as claimed in claim 18, wherein the position-fixing device further comprises an additional three-element switch, when the tissue is moved away either way from the second fixed position, the two three-element switches are turned off.
